(19) United States
(12) Patent Application Publication
(10) Pub. No.: US 2022/0401643 A1
(43) Pub. Date: Dec. 22, 2022

(54) DRUG PRODUCT CONTAINER AND DRUG DELIVERY SYSTEM

(71) Applicant: AMGEN INC., Thousand Oaks, CA (US)

(72) Inventors: Scott R. Gibson, Granada Hills, CA (US); Paul D. Faucher, Escondido, CA (US); Mehran Mojarrad, Thousand Oaks, CA (US)

(21) Appl. No.: 17/763,692

(22) PCT Filed: Oct. 14, 2020
(86) PCT No.: PCT/US20/55540

§ 371 Date: Mar. 25, 2022

Related U.S. Application Data
(60) Provisional application No. 62/924,027, filed on Oct. 21, 2019.
(60) Provisional application No. 62/925,692, filed on Oct. 24, 2019.

(51) Int. Cl. A61M 5/148

(52) U.S. Cl. CPC A61M 5/1483; A61M 2205/0238; A61M 2005/14513

(57) ABSTRACT

A drug delivery device includes a drug product container, a pressurized vessel, and an urging member. The drug product container can have at least one flexible wall and defining a cavity configured to contain a drug product. The pressurized vessel can contain a gas under pressure. And the urging member is in working connection with the pressurized vessel such that, upon at least partial release of the gas under pressure, the urging member moves from a first portion of the drug product container to a second portion, thereby ejecting at least a portion of the drug product from the drug product container.

### CROSS-REFERENCE TO RELATED APPLICATIONS

Priority is claimed to U.S. Provisional Patent Application No. 62/924,027, filed Oct. 21, 2019, and U.S. Provisional Patent Application No. 62/925,692, filed Oct. 24, 2019, the entire contents of each of which are hereby incorporated herein by reference.

### FIELD OF DISCLOSURE

The present disclosure generally relates to drug delivery containers and systems and, more particularly, to a drug product container and a system for long-term, continuous or semi-continuous, intravenous drug delivery.

### BACKGROUND

Drugs are administered to treat a variety of conditions and diseases. Intravenous (“IV”) therapy is a drug dosing process that delivers drugs directly into a patient's vein using an infusion contained in a delivery container such as an IV bag and tubing connected to a needle subsystem that fluidically communicates with the reservoir through the pump assembly collectively called an infusion set. Similarly, infusion therapy may encompass IV therapy and/or delivery to subcutaneous or other tissue. The term “IV” as used herein shall be used to refer to intravenous and/or infusion therapies. In IV therapies, drug dosings may be performed in a healthcare facility, or in some instances, at remote locations such as a patient's home. In certain applications, a drug delivery process may last for an extended period of time (e.g., for one hour or longer) or may include continuous or semi-continuous delivery of a drug over an extended period of time (e.g., for several hours, days, weeks, or longer). For many of these relatively long-term delivery requirements, a pump is often utilized to control and/or administer the drug to the patient. The pump may be coupled (physically, fluidly, and/or otherwise) to various components, such as a drug delivery container, supply lines, connection ports, and/or the patient.

It may be desirable to utilize a pump and/or overall system that is portable and/or wearable. It may also be desirable to utilize a pump and an overall system that minimizes patient inconvenience, minimizes the size and profile of the device and the overall system, minimizes the complexity of the device and overall system, minimizes the noise and vibration of the device, accommodates easy connection/disconnection and changeover of the infusion set, simplifies or automates priming of the line, accommodate easy delivery interruption and reestablishment based on required therapy and delivery profile, easily provides status of delivery and other important user information such as occlusion and volume of drug delivered or remaining in the reservoir, reduces the cost of the device and the overall system, increases the reliability and accuracy of the device and the overall system.

During use with a drug delivery system, a drug product container may become undesirably sealed, pinched, or otherwise permit drug product to become trapped within the drug product container. Undelivered drug product, known as “hold-up volume,” may be undesirable for several reasons, including: it may lead to inaccurate or undesirable dosage levels compromising drug efficacy, it may promote uncertainty in the drug delivery process, it may cause patient anxiety or frustration, it may require the patient or health care provider to squeeze or agitate the drug product container to release the hold-up volume, or it may lead to undesirable waste.

As described in more detail below, the present disclosure sets forth devices, systems, and methods for drug delivery embodying advantageous alternatives to existing devices, systems, and methods, and that may address one or more of the challenges or needs mentioned herein, as well as provide other benefits and advantages.

### BRIEF DESCRIPTION OF THE DRAWINGS

The above needs are at least partially met through provision of the systems and approaches for drug delivery device described in the following detailed description, particularly when studied in conjunction with the drawings, wherein:

FIG. 1 illustrates a plurality of exemplary drug product containers during “hold up volume” testing;

FIG. 2 illustrates an exemplary drug product container in accordance with various embodiments of the present disclosure;

FIGS. 3a and 3b illustrate an exemplary drug product container in accordance with various embodiments, in a storage configuration (FIG. 3a) and a dispensing configuration (FIG. 3b);

FIG. 4 illustrates two different exemplary drug product containers (top figures and bottom figures, respectively) in accordance with various embodiments, in a storage configuration and a dispensing configuration (left and right figures, respectively);

FIG. 5 illustrates an exemplary drug product container in accordance with various embodiments, in a storage configuration (left) and a dispensing configuration (right);

FIG. 6 illustrates additional exemplary drug product containers in accordance with various embodiments;

FIG. 7 illustrates another exemplary drug product container in accordance with various embodiments of the present disclosure;

FIG. 8 illustrates a cross-sectional view taken through line I-I of FIG. 7 of an exemplary drug product container in accordance with various embodiments;

FIG. 9 illustrates a cross-sectional view taken through line I-I of FIG. 7 of another exemplary drug product container in accordance with various embodiments;

FIG. 10 illustrates a cross-sectional view taken through line I-I of FIG. 7 of another exemplary drug product container in accordance with various embodiments; and

FIG. 11 illustrates a perspective view of an exemplary drug delivery assembly in accordance with various embodiments.

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments. It will further be appreciated that certain actions and/or steps may be described or depicted in a particular order of occurrence while those skilled in the art will understand that such specificity with respect to sequence is not actually required. It will also be understood that the terms and expressions used herein have the ordinary technical meaning as is accorded to such terms and expressions by persons skilled in the technical field as set forth above except where different specific meanings have otherwise been set forth herein.

### GENERAL DESCRIPTION

The present disclosure relates to a drug product container and/or drug delivery device and, more particularly, to a drug product container and a drug delivery assembly or system for long-term, continuous or semi-continuous, intravenous drug delivery. Under some conditions, a drug delivery process may last for an extended period of time (e.g., for one hour or longer) or may include continuous or semi-continuous delivery of a drug over an extended period of time (e.g., for several hours, days, weeks, or longer) or may include delivery via an intravenous connection to a patient. The present disclosure utilizes various features, devices, systems, and methods to prevent drug product from becoming trapped within the drug product container (minimize hold-up volume).

In some aspects, the present disclosure includes a drug delivery device comprising: a drug product container having at least one flexible wall and defining a cavity configured to contain a drug product; a pressurized vessel containing a gas under pressure; an urging member in working connection with the pressurized vessel such that, upon at least partial release of the gas under pressure, the urging member moves from a first portion of the drug product container to a second portion, thereby ejecting at least a portion of the drug product from the drug product container.

The drug delivery device may include mechanical flat leaf springs that are applied on the container and designed to apply higher pressure at the distal end of the container to squeeze the drug out. The drug delivery device may include a spring-loaded solid roller mechanism used to progressively apply mechanical pressure on the bag starting from distal end and progress towards the proximal end where drug is dispensed through exit port. The drug delivery device may include a roller element guided in slots at two ends to maintain a straight path for linear movement. Low friction elements such as ball bearings or low-friction materials could be used to enable smooth movement of the roller in the guide slots on the sides of the roller. The roller element may be actuated with a torsion spring to rotate and pull the bag from the distal end towards the proximal end where the fluid exit port is located. Additionally or alternatively, With a driven roller system, the bag will be engaged with the rollers starting at the distal end and the bag will be pulled by the rollers toward the proximal end or the rollers will roll along the bag from the distal end to the proximal end where the exit port is. In this configuration a secondary idle roller on the opposite side of the bag ensures uniform pressure applied on the bag by sandwiching it to squeeze the fluid out as the rollers turn while minimizing residual volume remaining in the flexible drug container bag.

The drug delivery device may include component(s) for pneumatically urging a compressed gas supply. When gas is released, the pressure applies force on a rotating impeller connected to the roller element to rotate the roller to pull the bag and expel the drug out. Similar to that described above, there is an idler roller on the opposite side of the bag to ensure uniform pressure on both sides. The drug delivery device may include a secondary inflatable bag that is used to apply pressure progressively from distal end to proximal end to encourage the drug out while minimizing premature collapse or blockage of the bag to ensure minimal residual volume in the bag. The secondary bag may be fashioned in a serpentine pattern starting with larger tubular elements at the distal end of the primary bag and progressively reduce in diameter towards proximal end of the primary bag. This approach ensures higher pressures at the distal end of the primary container to ensure drug is squeezed out towards the proximal end where the exit port for dispensing is located.

Currently drug delivery systems in handheld or wearable format utilize a number of different actuations such as electromechanical, pneumatic, hydraulic, mechanical springs, chemical reaction chambers, osmotic pressure, electrochemical cells, expandable gel matrix, etc. to name a few. However, to reduce the cost and/or complexity of an overall device for a bolus delivery of medication, non-electronic approaches present an attractive alternative. Among non-electronic options for actuation, helical compression springs may be utilized on both handheld and wearable drug delivery systems. However, these options present challenges as they tend to have a wide-ranging tolerance and cannot be applied to variety of liquid medications with varying fluid viscosities for subcutaneous delivery. On the other hand, compressed gas primary source of power provides a large initial actuation force capable of handling high viscosity medium while delivering at high injection rate.

This disclosure includes utilizing compressed gas such as CO2 in pressurizing a flexible bag primary drug container in a manner not to cause any inadvertent fluid blockage. For example, if the flexible bag drug container is directly exposed to high-pressure gas such as CO2, there is a possibility that the bag may collapse prematurely at points of lowest resistance causing blockage of continuous flow of medication out of the bag which results in large residual volumes remaining and compromising drug efficacy by not delivering the intended full volume of the drug.

Typically, the distal end of a component or of a device is to be understood as meaning the end furthest (along the fluid path) from the user's body and the proximal end is to be understood as meaning the end closest (along the fluid path) to the user's body. Likewise, in this application, the “distal direction” is to be understood as meaning the direction away from the user's body, and the “proximal direction” is to be understood as meaning the direction toward the user's body. In the context of the drug product container disclosed herein, the port that leads to the pressurized gas is the distal port (e.g., the distal end of the bag) of the drug product container and the port that leads to the patient is at the proximal port (e.g. the proximal end of the bag) of the drug product container.

In some other aspects, the present disclosure may include a container for a drug product comprising: a first wall and a second wall cooperating to define a cavity configured to contain a drug product; and an outlet in fluid communication with the cavity to selectively permit the drug product to exit the cavity. At least a portion of at least the first wall or the second wall may include an anti-sealing component that resists sealing between the first wall and the second wall while the drug product exits the cavity.

For example, the anti-sealing component may be a ridge or a groove or pattern of ridges and grooves across the surface. The container may have one or both walls with the anti-sealing portion. Alternatively texturing the surfaces in contact with drug solution may take the form of micro structured patters with precise dimensions and features to allow for most optimal microfluidic networks to prevent any premature collapse under high vacuum or applied pressure to the drug reservoir.

In some aspects, the drug product container may be a flexible, non-pressurized drug reservoir (e.g., an IV bag) with anti-sealing and/or anti-pinching features that allow it to continue to dispense fluid when folded, creased, or pinched by preventing the bag from sealing against itself. This may prevent or mitigate the formation of pouches of fluid that are hydraulically isolated from the pump, thereby ensuring that the pump can reliably dispense the entire contents of the reservoir.

These anti-sealing and/or anti-pinching features may take the form of:
Grooves or other texture/profile present on the inner surface(s) of the reservoir,
A separate, textured and/or porous panel member placed between the inner surfaces of the reservoir, or
Other suitable components.

Any of the disclosed drug containers may be used in a system, such as with a pump mechanism (e.g., a peristaltic pump) and a fluid path. The pump is preferably compact and attaches directly or remotely to the drug container. The pump is also preferably able to overcome differences in pressure between the fluid in the reservoir and the patient's intravenous or subcutaneous tissue (due to differences in elevation, atmospheric pressure, and/or other external forces).

The system may also include a means to attach the device to the patient's body (e.g. a belt clip, an elastic strap, a Velcro strap, a waist or shoulder pack, backpack, or similar). The flexible reservoir and pump housing may be attached separately and in different locations on the patient's body while maintaining a hydraulic connection via the fluid tubing (IV line set). For example, the flexible reservoir may be placed in a garment pocket while the pump is clipped to the patient's belt. The drug product container and/or the system may provide several potential advantages compared to existing designs, especially when used in portable applications, such as:
The anti-pinch/anti-seal features in the flexible reservoir allow reliable drug delivery even if the flexible reservoir is folded, creased, or pinched,
The anti-sealing and/or anti-pinching features of the flexible reservoir eliminate the need for a rigid enclosure around the flexible reservoir (to prevent folding or pinching), which enhances patient comfort during use,
The flexibility of the durable pump controller with attached pump head assembly enables a wide variety of attachment methods to be used to affix the device to the patient's body,
The flexibility of the reservoir enables a wide variety of attachment locations on the patient's body,
The anti-pinch/anti-seal feature would prevent premature collapse of the reservoir especially when the vacuum pressure is high to allow for fast delivery,
The anti-sealing/anti-pinching feature would enable the use of alternative actuations mechanisms such as high-pressure gas cartridges or other mechanical means such as pressure-plates to squeeze the reservoir as opposed to vacuum pressure to extract fluid out.

During use and/or preparation for use, the IV infusion set may be initially separate from the flexible reservoir (IV bag). The IV infusion set tubing is attached to the flexible reservoir, then primed and the attached pump head assembly is connected to the pump controller. The proximal end of the IV infusion set is connected to the patient and the pump controller is activated. The pump controller with attached IV infusion set and reservoir can then be attached to the patient via one of the attachment methods described. The method of attachment is flexible based on the patient's needs and preferences. Because of the “anti-pinch” features in the IV bag, the pump controller can deliver the full contents of the IV bag regardless of where and how the IV bag is carried by the patient.

### DETAILED DESCRIPTION OF THE FIGURES

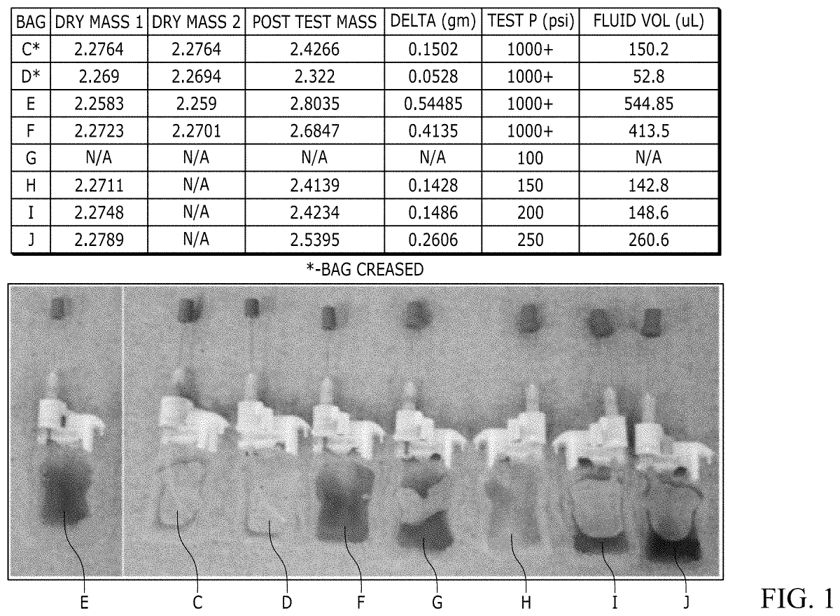

FIG. 1 shows various small volume flexible bag containers (e.g., bags) C through J, each in direct contact with high-pressure gas. As shown there is premature collapse of the bags C though J at elevated stress concentration areas when exposed to greater than 150 psi CO2 pressure. For example, FIG. 1 shows representative 2.5 mL primary container bags C through J showing different amounts of residual volumes of 54 cP fluid medium left when exposed to various gas pressures. The table in FIG. 1 displays the relative data for each bag C through J tested. There are several ways to address this challenge as described herein.

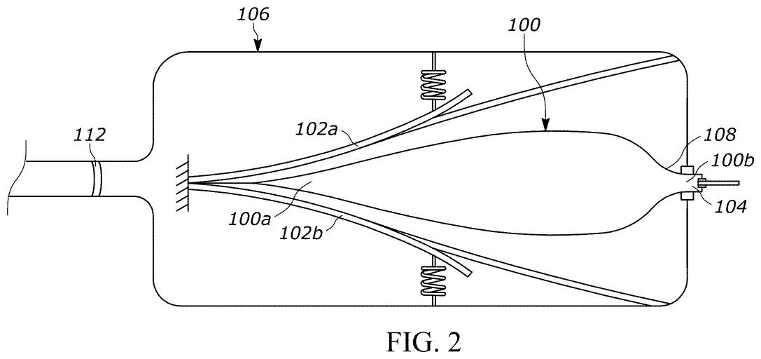

FIG. 2 shows flat leaf springs 102a, 102b, collectively acting as an urging member, utilized to compress a bag 100 from a bottom end 100a proceeding forward towards an exit port 104 as a gas is released into an enclosure 106 via a high pressure gas port 112, which also triggers release of the leaf springs 102a, 102b on either side of the bag 100. In this example the pair of flat leaf springs 102a, 102b are arranged in a V-configuration as shown in FIG. 2 and apply a preload to both sides of the bag 100. When the high-pressure gas is released in to a sealed enclosure 106 holding the bag 100, the flat springs 102a, 102b are released to apply additional pressure starting from the bottom 100a of the bag 100 working its way up to a neck 108 at a top 100b of the bag 100.

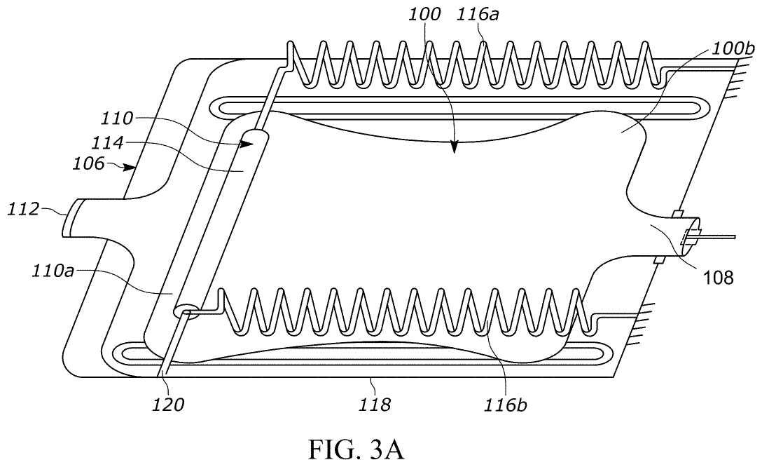

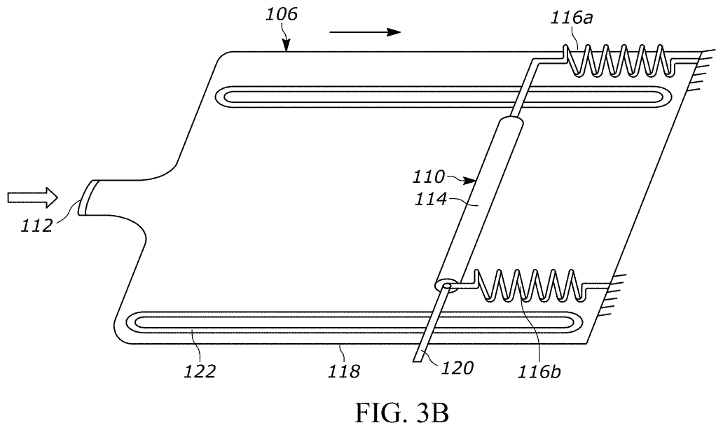

FIGS. 3a and 3b is another version showing a spring-loaded cylindrical roller mechanism 110, serving as an urging member, that is activated when gas is released via the high pressure gas port 112 into the enclosure 106 holding the primary container bag 100. The roller mechanism 110 includes a roller 114 that sweeps across the bag 100 starting from the bottom 110a of the bag 100 working its way towards the neck 108 at the top 100b of the bag 100 similar to previous concept as shown in FIG. 2. The actuation could also be via SMA wire, extension springs or other mechanical or electromechanical means. FIG. 3a shows the initial state before trigger and FIG. 3b shows final (or at least a non-final post-trigger) state after trigger. As a more specific example, the roller concept shown in FIGS. 3a and 3b takes advantage of squeezing the bag 100 from the bottom 100a of the bag 100 by moving a roller 114 that applies constant pressure on the bag 100 and moves by extension springs 116a, 116b or other mechanical actuators towards the top 100b of the bag 100. The roller release is triggered by the gas release into the enclosure 106 via the high pressure gas port 112. As also seen in FIGS. 3a and 3b, the enclosure 106 can have a rigid flat bottom wall 118 and a sliding rod 120 disposed in a slot 122 (see FIGS. 3b) of the enclosure 106 can move with the roller 114 as it applies pressure across the bag 100.

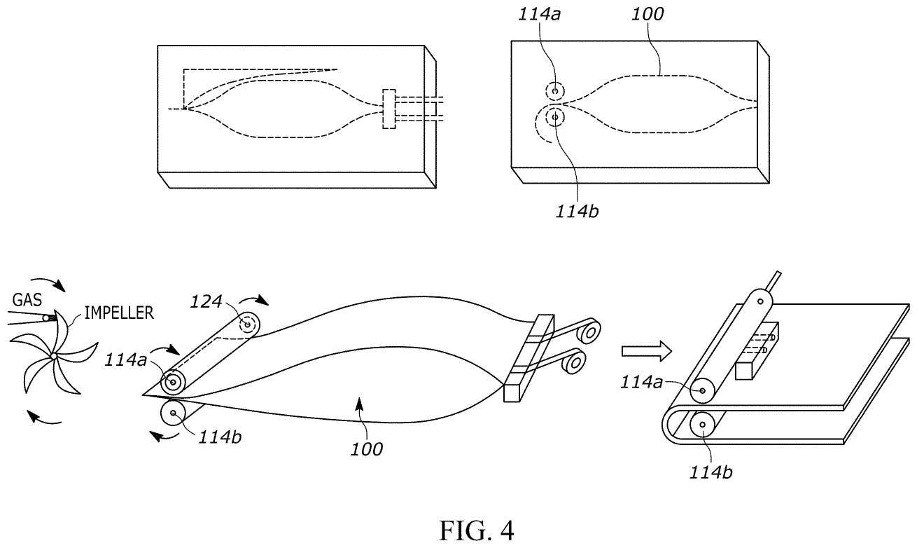

FIG. 4 shows another exemplary roller mechanism 110, serving as an urging member, but instead of the roller 114 moving to squeeze the bag 100, the bag 100 moves as a pair of stationary rollers 114a, 114b may be turned either with use of a driver 124 such as torsional springs, electromotor or pneumatic impeller as shown in FIG. 4. As a more specific example, only one roller 114a needs to rotate, being driven by the driver 124, while the other roller 114b will be an idler roller to ensure uniform pressure applied on the bag 100 from both sides. The rolled bag 100 is then collected below as the fluid is dispensed out. The example shown in FIG. 4 uses a drive roller 114a to pull the bag 100 while a second idle roller 114b on the other side is used to ensure uniform pressure on the bag 100 while the bag 100 is being pulled in.

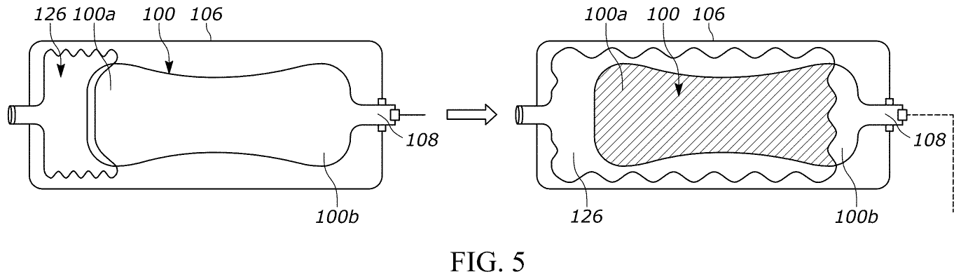

FIG. 5 shows a secondary bag 126, serving as an urging member, also disposed in the enclosure 106 (e.g., pressure chamber) and which can be utilized to isolate the high-pressure gas from the primary container bag 100 to apply indirect pressure to the primary container bag 100 to squeeze the drug out. The secondary bag 126 is fluidly coupled to the high pressure gas port 112. When the gas is released into the secondary bag 126 it inflates in a manner that will envelop the primary drug container bag 100 starting from bottom 100a of the bag 100 and working its way up towards the neck 108 at the top 100b of the bag 100. For example, the inflatable secondary bag 126 envelops the primary bag 100 when inflated by the high-pressure gas, as can be seen on the right side of FIG. 5. The inflation starts near the bottom 100a of the primary bag 100 and grows moving forward to be the top 100b, thereby applying pressure onto the primary bag 100 to ensure minimum residual volume.

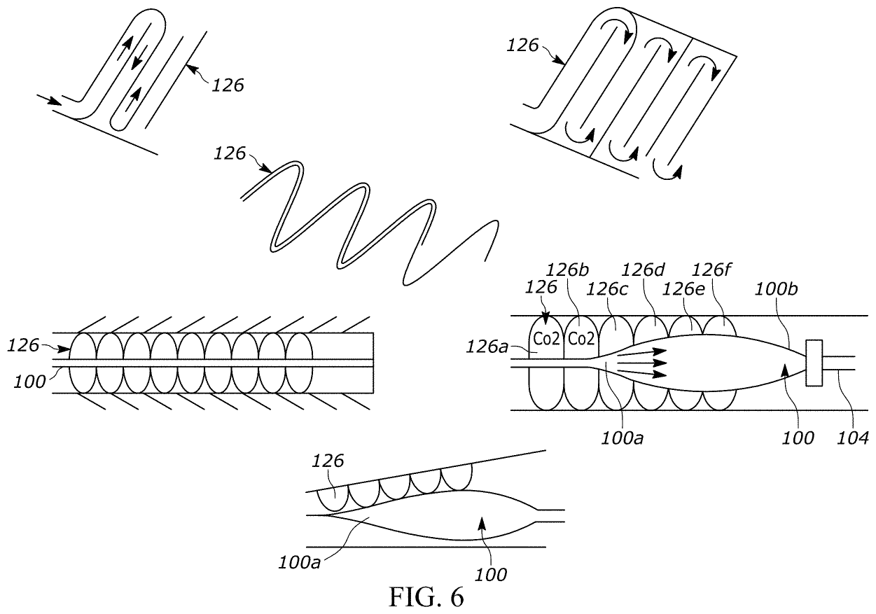

FIG. 6 shows different versions of an inflatable secondary bag 126, serving as an urging member, applying higher pressure closer to the bottom 100a of the primary drug container bag 100 with gradually decreasing pressure towards the manifold or top 100b of the primary container bag 100. The design takes advantage of a secondary bag 126 with a tubular structure fashioned in a serpentine pattern with a variety of sections 126a through 126f, in one depicted version for example, where the section 126a located closest to the bottom 100a of the bag 100 has the largest diameter tubing and the section located closest to the top 100b of the bag 100 has the smallest diameter tubing, with the intervening sections 126b through 126e decreasing in diameter from the largest section 126a to the smallest section 126f. For example, in the Inflatable serpentine shaped secondary tubular bag 126 of FIG. 6, whereby higher pressures are applied in larger diameter tubes at the bottom 100a of the bag 100 compared to smaller diameter tubes at the top 100b of the bag 100, the gas supplied to the secondary bag via the high pressure gas port 112 causes the secondary bag 126 to apply a gradually decreasing pressure to the primary drug container bag 100 starting from the bottom 100a of the primary drug container bag 100 and proceeding towards the top 100b of the bag 100 at the exit port 104.

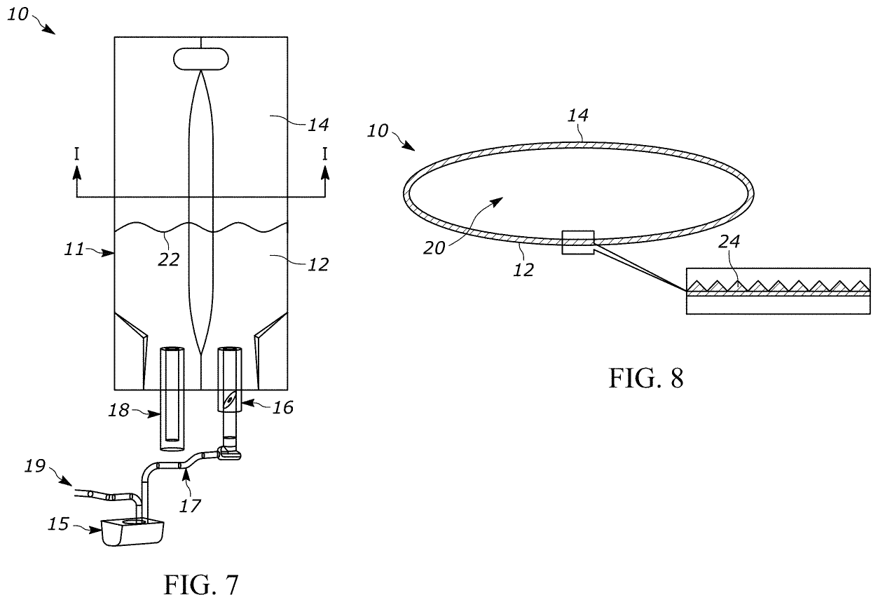

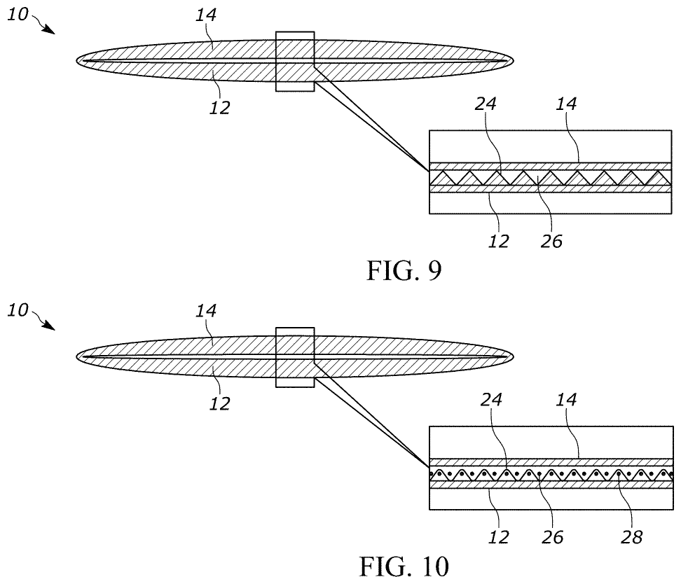

Turning to FIG. 7, another version of a drug product container 10 in accordance with the present disclosure can include a flexible reservoir 11 have a first wall 12, a second wall 14, and an outlet 16. The outlet 16 can be coupled to a pump head 15 via a first tubing set 17, and the pump head 15 can communicate with a patient via a second tubing set 19. The container 10 described and depicted could be implemented as the bag 100 of any of the disclosure related to FIGS. 1-6 above. The container 10 may include an additional inlet/outlet 18. The first and second walls 12, 14 cooperate to define a cavity 20 (seen in FIG. 8) for selectively containing a drug product 22. As shown in FIG. 8, at least one of the first and second walls 12, 14 includes an anti-sealing component 24 such as a plurality of grooves, protrusions, or other suitable features on the inner surface thereof. The anti-sealing components 24 on one wall may cooperate with the opposing wall to define fluid flow channels 26 (seen in FIGS. 9 and 10) when collapsed to facilitate fluid flow by creating pathways for the drug product to migrate towards the outlet 16 of the container 10. In other versions, such as that depicted in FIG. 10, one or more of the walls 12, 14 may also include an anti-pinch panel member 28 disposed on the internal surface thereof that may allow for the bag to fold or distort but not pinch and prevent fluid flow.

The drug product container 10 may have the anti-sealing components 24 on one of the two walls 12, 14; both of the walls 12, 14; or on select portions of one or both walls 12, 14. In the case where both walls 12, 14 have the anti-sealing components 24, it may be preferable for the anti-sealing components 24 to have different orientations so that the opposing ridges do not seal with each other and/or form pockets for fluid to gather. It may also be desirable to use a material having low surface friction. It may also be desirable to have surface treatments of walls 12 and 14 to increase hydrophobicity of the walls 12 and 14 to ensure no amount of drug solution adheres to the walls 12, 14. This is especially effective for drug solution with high surface tension properties.

The drug product container 10 may be formed of a single component that is folded and sealed on itself or it may be formed from two or more panels that are sealed together. Similarly, the anti-sealing components 24 may be integrally formed in one of the walls or they may be formed on a separate component (e.g. panel) that is then coupled with one or both of the walls or positioned within the cavity 20. In any case, the material forming the anti-sealing components 24 is preferably compatible with the drug product contained therein and is sterilized before use.

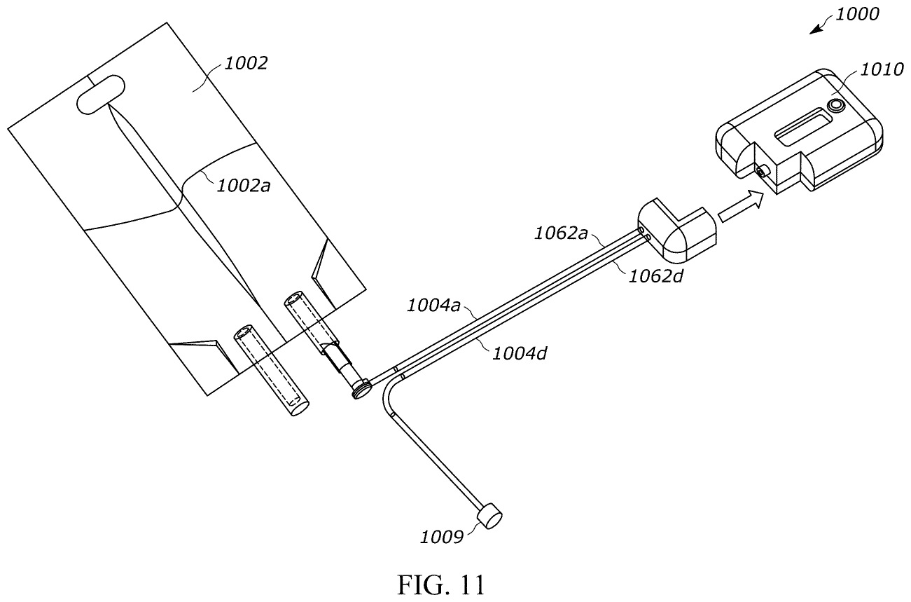

FIG. 11 shows an exemplary drug delivery assembly 1000 (or “system”) for use with the container 10 of FIGS. 7-10, for example, or any of the bags 100 disclosed in FIGS. 1-6. For example, the assembly 1000 shown in FIG. 11 includes a drug product container 1002 for containing a drug product 1002a (or medicament), an IV input line 1004a, an IV output line 1004d, an air vent 1009 for purging air from the fluid flowpath, and tubing portions 1062a, 1062d leading to and from the pump 1010. As a more specific example, the connection points may include quick-connect sterile connectors with respective sub-components that selectively mate with each other while maintaining sterility or another desirable cleanliness standard. For example, the quick-connect sterile connectors may snap or twist or screw together; they may have sheathed or covered components that become unsheathed or uncovered upon connection; and/or they may have Luer Lock or modified Luer Lock configurations. As another example, the connectors may include one or more stake connectors for coupling one of the tube portions 1062a with an IV bag. The distal end of the IV output line 1004d may also include or be coupled with a drug delivery connector (not shown) such as a needle, a luer lock component, or another suitable component. An IV spike may pierce the port of the drug container 1002 to physically connect the drug product container to the fluid path assembly 1060.

It may be desirable to utilize components that allow for fast/easy/sterile connections/disconnections. The fluid flowpath may be defined by a sterile single-use tubing system and valve system. The system may be used to provide intravenous, subcutaneous, intra-arterial, intramuscular, and/or epidural delivery approaches. By using the system, patient anxiety and or confusion may be reduced due to reduced preparation complexity and wait times caused by the drug preparation process.

In some examples, the system may be utilized with medicament in the form of a half-life extended bispecific T cell engager (BiTE). For example, the active pharmaceutical ingredient (“API”) may be between approximately 2 mcg and approximately 100 mcg, depending on the BiTE and container size, which may be in a powdered form (i.e., lyophilized) requiring reconstitution. In other examples, the drug product may be in liquid form and may not require reconstitution. Nonetheless, the system includes an accurate quantity of drug product, and thus does not require the need to add additional quantities thereto in a sterile environment. In some examples, the API may be in the form of a half-life extended (“HLE”) BiTE and/or an IV-admin monoclonal antibody (“mAbs) as desired. These HLE BiTEs include an antibody Fc region that advantageously provides different drug properties such as longer and extended half-lives. Accordingly, such APIs may be preferred due to their ability to maintain protective levels in the patient for relatively longer periods of time. Nonetheless, in other examples, the API may be in the form of a canonical-BiTE that is to be administered in a professional healthcare environment.

The drug product container may be in the form of an IV bag, a vial, a prefilled syringe, or similar container that includes a reconstitution container body defining an inner volume. The inner volume may be sterile. In some approaches, the reconstitution container adapter may also be a CSTD (or, in examples where the prefilled reconstitution container is in the form of a syringe, the container adapter may be a needle) that mates, engages, and/or couples to the vial adapter. Additionally or alternatively, the drug product can be bulk lyophilized and filled into a cartridge or container that is typically used to administer with an IV pump. If needed, the dehydrated forms of IVSS, NaCl, and any other components needed for the final administered solution can be bulk lyophilized and filled into the cassette for long term storage.

As previously noted, in some examples, the prefilled drug product container may be in the form of a prefilled syringe that contains the drug product. In these examples the drug product may be in the form of a liquid BiTE formulation used in conjunction with a monoclonal antibody (mAb), In these examples, the drug product may be directly added to the delivery container without the use of a vial adapter system (such as the above-mentioned CSTDs) where more traditional needle-syringe injection/delivery into the container is preferred, which may advantageously simplify and/or improve supply chain and manufacturing control, and may further allow for more compact commercial packaging that takes up less space in storage systems at healthcare facilities. In these examples, the prefilled drug product vial may or may not need to be reconstituted prior to transferring the drug product to the delivery container.

The system may be distributed and/or sold as a common kit packaging, but other suitable distribution/packaging is suitable. The drug product may be in the form of a half-life extended bispecific T cell engager (BiTE), but other drug products are suitable. The diluent may be water for injection (“WFI”), but other diluents may be suitable. The containers may be pliable bags, such as IV bags, but other containers may be suitable. In some examples, one or more of the containers is in the form of an IV drip bag constructed from a polymer or other material, e.g., 250 mL 0.9% Sodium Chloride IV bag constructed of a suitable material such as polyolefin, non-DEHP (diethylhexl phthalate), PVC, polyurethane, or EVA (ethylene vinyl acetate) and can be filled to a volume of approximately 270 mL to account for potential moisture loss over long-term storage.

In some examples, the prefilled delivery container is in the form of an IV bag constructed from a polymer or other material, e.g., 250 mL 0.9% Sodium Chloride IV bag constructed of a suitable material such as polyolefin, non-DEHP (diethylhexl phthalate), PVC, polyurethane, or EVA (ethylene vinyl acetate) and can be filled to a volume of approximately 270 mL to account for potential moisture loss over long-term storage. Other examples of suitable delivery containers are possible such as, for example, a glass bottle or container. Example suitable prefilled delivery containers are described in U.S. application Ser. No. 62/804,447, filed on Feb. 12, 2019 and U.S. application Ser. No. 62/877,286 filed on Jul. 22, 2019, the contents of each of which are incorporated by reference in their entirety.

The above description describes various devices, assemblies, components, subsystems and methods for use related to a drug delivery device. The devices, assemblies, components, subsystems, methods or drug delivery devices can further comprise or be used with a drug including but not limited to those drugs identified below as well as their generic and biosimilar counterparts. The term drug, as used herein, can be used interchangeably with other similar terms and can be used to refer to any type of medicament or therapeutic material including traditional and non-traditional pharmaceuticals, nutraceuticals, supplements, biologics, biologically active agents and compositions, large molecules, biosimilars, bioequivalents, therapeutic antibodies, polypeptides, proteins, small molecules and generics. Non-therapeutic injectable materials are also encompassed. The drug may be in liquid form, a lyophilized form, or in a reconstituted from lyophilized form. The following example list of drugs should not be considered as all-inclusive or limiting.

The drug will be contained in a reservoir. In some instances, the reservoir is a primary container that is either filled or pre-filled for treatment with the drug. The primary container can be a vial, a cartridge or a pre-filled syringe.

In some embodiments, the reservoir of the drug delivery device may be filled with or the device can be used with colony stimulating factors, such as granulocyte colony-stimulating factor (G-CSF). Such G-CSF agents include but are not limited to Neulasta® (pegfilgrastim, pegylated filgastrim, pegylated G-CSF, pegylated hu-Met-G-CSF) and Neupogen® (filgrastim, G-CSF, hu-MetG-CSF), UDENYCA® (pegfilgrastim-cbqv), Ziextenzo® (LA-EP2006; pegfilgrastim-bmez), or FULPHILA (pegfilgrastim-bmez).

In other embodiments, the drug delivery device may contain or be used with an erythropoiesis stimulating agent (ESA), which may be in liquid or lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin iota, epoetin omega, epoetin delta, epoetin zeta, epoetin theta, and epoetin delta, pegylated erythropoietin, carbamylated erythropoietin, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RANKL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“IL1-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific IgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 145c7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like;Hepatocyte growth factor (“HGF”) specific antibodies, peptibodies, related proteins, and the like, including those that target the HGF/SF:cMet axis (HGF/SF:c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40L specific antibodies, peptibodies, related proteins, and the like, including but not limited to proteins that bind OX40L and/or other ligands of the OX40 receptor; Activase® (alteplase, tPA); Aranesp® (darbepoetin alfa) Erythropoietin [30-asparagine, 32-threonine, 87-valine, 88-asparagine, 90-threonine], Darbepoetin alfa, novel erythropoiesis stimulating protein (NESP); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-α4β7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Kanjinti™ (trastuzumab-anns) anti-HER2 monoclonal antibody, biosimilar to Herceptin®, or another product containing trastuzumab for the treatment of breast or gastric cancers; Humatrope® (somatropin, Human Growth Hormone); Humira® (adalimumab); Vectibix® (panitumumab), Xgeva® (denosumab), Prolia® (denosumab), Immunoglobulin G2 Human Monoclonal Antibody to RANK Ligand, Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Soliris™ (eculizumab); pexelizumab (anti-C5 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Orthoclone OKT3® (muromonab-CD3, anti-CD3 monoclonal antibody); Procrit® (epoetin alfa); Remicade® (infliximab, anti-TNFα monoclonal antibody); Reopro® (abciximab, anti-GP Ilb/Ilia receptor monoclonal antibody); Actemra® (anti-IL6 Receptor mAb); Avastin® (bevacizumab), HuMax-CD4 (zanolimumab); Mvasi™ (bevacizumab-awwb); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 145c7-CHO (anti-IL15 antibody, see U.S. Pat. No. 7,153,507); Tysabri® (natalizumab, anti-α4integrin mAb); Valortim® (MDX-1303, anti-B. anthracis protective antigen mAb); ABthrax™; Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2Rα mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNFα mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-α5β1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD4OL mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-ganglioside GM2 mAb; anti-GDF-8 human mAb (MYO-029); anti-GM-CSF Receptor mAb (CAM-3001); anti-HepC mAb (HuMax HepC); anti-IFNα mAb (MEDI-545, MDX-198); anti-IGF1R mAb; anti-IGF-1R mAb (HuMax-Inflam); anti-IL12 mAb (ABT-874); anti-IL12/IL23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); BMS-66513; anti-Mannose Receptor/hCGβ mAb (MDX-1307); anti-mesothelin dsFv-PE38 conjugate (CAT-5001); anti-PD1mAb (MDX-1106 (ONO-4538)); anti-PDGFRα antibody (IMC-3G3); anti-TGFβ mAb (GC-1008); anti-TRAIL Receptor-2 human mAb (HGS-ETR2); anti-TWEAK mAb; anti-VEGFR/Flt-1 mAb; and anti-ZP3 mAb (HuMax-ZP3).

In some embodiments, the drug delivery device may contain or be used with a sclerostin antibody, such as but not limited to romosozumab, blosozumab, BPS 804 (Novartis), Evenity™ (romosozumab-aqqg), another product containing romosozumab for treatment of postmenopausal osteoporosis and/or fracture healing and in other embodiments, a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716; NV1020; NV12023; NV1034; and NV1042. In some embodiments, the drug delivery device may contain or be used with endogenous tissue inhibitors of metalloproteinases (TIMPs) such as but not limited to TIMP-3. In some embodiments, the drug delivery device may contain or be used with Aimovig® (erenumab-aooe), anti-human CGRP-R (calcitonin gene-related peptide type 1 receptor) or another product containing erenumab for the treatment of migraine headaches. Antagonistic antibodies for human calcitonin gene-related peptide (CGRP) receptor such as but not limited to erenumab and bispecific antibody molecules that target the CGRP receptor and other headache targets may also be delivered with a drug delivery device of the present disclosure. Additionally, bispecific T cell engager (BiTE®) antibodies such as but not limited to BLINCYTO® (blinatumomab) can be used in or with the drug delivery device of the present disclosure. In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure. In some embodiments, the drug delivery device may contain or be used with Avsola™ (infliximab-axxq), anti-TNF α monoclonal antibody, biosimilar to Remicade® (infliximab) (Janssen Biotech, Inc.) or another product containing infliximab for the treatment of autoimmune diseases. In some embodiments, the drug delivery device may contain or be used with Kyprolis® (carfilzomib), (2S)-N-((S)-1-((S)-4-methyl-1((R)-2-methyloxiran-2-yl)-1-oxopentan-2-ylcarbamoyl)-2-phenylethyl)-2-((S)- 2-(2-morpholinoacetamido)-4-phenylbutanamido)-4-methylpentanamide, or another product containing carfilzomib for the treatment of multiple myeloma. In some embodiments, the drug delivery device may contain or be used with Otezla® (apremilast), N-[2-[(1S)-1-(3-ethoxy-4-methoxyphenyl)-2-(methylsulfonyl)ethyl]-2,3-dihydro-1,3-dioxo-1H-isoindol-4-yl]acetamide, or another product containing apremilast for the treatment of various inflammatory diseases. In some embodiments, the drug delivery device may contain or be used with Parsabiv™ (etelcalcetide HCl, KAI-4169) or another product containing etelcalcetide HCl for the treatment of secondary hyperparathyroidism (sHPT) such as in patients with chronic kidney disease (KD) on hemodialysis. In some embodiments, the drug delivery device may contain or be used with ABP 798 (rituximab), a biosimilar candidate to Rituxan®/MabThera™, or another product containing an anti-CD20 monoclonal antibody. In some embodiments, the drug delivery device may contain or be used with a VEGF antagonist such as a non-antibody VEGF antagonist and/or a VEGF-Trap such as aflibercept (Ig domain 2 from VEGFR1 and Ig domain 3 from VEGFR2, fused to Fc domain of IgG1). In some embodiments, the drug delivery device may contain or be used with ABP 959 (eculizumab), a biosimilar candidate to Soliris®, or another product containing a monoclonal antibody that specifically binds to the complement protein C5. In some embodiments, the drug delivery device may contain or be used with Rozibafusp alfa (formerly AMG 570) is a novel bispecific antibody-peptide conjugate that simultaneously blocks ICOSL and BAFF activity. In some embodiments, the drug delivery device may contain or be used with Omecamtiv mecarbil, a small molecule selective cardiac myosin activator, or myotrope, which directly targets the contractile mechanisms of the heart, or another product containing a small molecule selective cardiac myosin activator. In some embodiments, the drug delivery device may contain or be used with Sotorasib (formerly known as AMG 510), a KRAS^{G12C} small molecule inhibitor, or another product containing a KRAS^{G12C} small molecule inhibitor. In some embodiments, the drug delivery device may contain or be used with Tezepelumab, a human monoclonal antibody that inhibits the action of thymic stromal lymphopoietin (TSLP), or another product containing a human monoclonal antibody that inhibits the action of TSLP. In some embodiments, the drug delivery device may contain or be used with AMG 714, a human monoclonal antibody that binds to Interleukin-15 (IL-15) or another product containing a human monoclonal antibody that binds to Interleukin-15 (IL-15). In some embodiments, the drug delivery device may contain or be used with AMG 890, a small interfering RNA (siRNA) that lowers lipoprotein(a), also known as Lp(a), or another product containing a small interfering RNA (siRNA) that lowers lipoprotein(a). In some embodiments, the drug delivery device may contain or be used with ABP 654 (human IgG1 kappa antibody), a biosimilar candidate to Stelara®, or another product that contains human IgG1 kappa antibody and/or binds to the p40 subunit of human cytokines interleukin (IL)-12 and IL-23. In some embodiments, the drug delivery device may contain or be used with Amjevita™ or Amgevita™ (formerly ABP 501) (mab anti-TNF human IgG1), a biosimilar candidate to Humira®, or another product that contains human mab anti-TNF human IgG1. In some embodiments, the drug delivery device may contain or be used with AMG 160, or another product that contains a half-life extended (HLE) anti-prostate-specific membrane antigen (PSMA)xanti-CD3 BiTE® (bispecific T cell engager) construct. In some embodiments, the drug delivery device may contain or be used with AMG 119, or another product containing a delta-like ligand 3 (DLL3) CART (chimeric antigen receptor T cell) cellular therapy. In some embodiments, the drug delivery device may contain or be used with AMG 119, or another product containing a delta-like ligand 3 (DLL3) CART (chimeric antigen receptor T cell) cellular therapy. In some embodiments, the drug delivery device may contain or be used with AMG 133, or another product containing a gastric inhibitory polypeptide receptor (GIPR) antagonist and GLP-1R agonist. In some embodiments, the drug delivery device may contain or be used with AMG 171 or another product containing a Growth Differential Factor 15 (GDF15) analog. In some embodiments, the drug delivery device may contain or be used with AMG 176 or another product containing a small molecule inhibitor of myeloid cell leukemia 1 (MCL-1). In some embodiments, the drug delivery device may contain or be used with AMG 199 or another product containing a half-life extended (HLE) bispecific T cell engager construct (BITE®). In some embodiments, the drug delivery device may contain or be used with AMG 256 or another product containing an anti-PD-1xIL21 mutein and/or an IL-21 receptor agonist designed to selectively turn on the Interleukin 21 (IL-21) pathway in programmed cell death-1 (PD-1) positive cells. In some embodiments, the drug delivery device may contain or be used with AMG 330 or another product containing an anti-CD33xanti-CD3 BiTE® (bispecific T cell engager) construct. In some embodiments, the drug delivery device may contain or be used with AMG 404 or another product containing a human anti-programmed cell death-1(PD-1) monoclonal antibody being investigated as a treatment for patients with solid tumors. In some embodiments, the drug delivery device may contain or be used with AMG 427 or another product containing a half-life extended (HLE) anti-fms-like tyrosine kinase 3 (FLT3)xanti-CD3 BiTE® (bispecific T cell engager) construct. In some embodiments, the drug delivery device may contain or be used with AMG 430 or another product containing an anti-Jagged-1 monoclonal antibody. In some embodiments, the drug delivery device may contain or be used with AMG 506 or another product containing a multi-specific FAPx4-1BB-targeting DARPin® biologic under investigation as a treatment for solid tumors. In some embodiments, the drug delivery device may contain or be used with AMG 509 or another product containing a bivalent T-cell engager and is designed using XmAb® 2+1 technology. In some embodiments, the drug delivery device may contain or be used with AMG 562 or another product containing a half-life extended (HLE) CD19xCD3 BiTE® (bispecific T cell engager) construct. In some embodiments, the drug delivery device may contain or be used with Efavaleukin alfa (formerly AMG 592) or another product containing an IL-2 mutein Fc fusion protein. In some embodiments, the drug delivery device may contain or be used with AMG 596 or another product containing a CD3xepidermal growth factor receptor vIII (EGFRvIII) BiTE® (bispecific T cell engager) molecule. In some embodiments, the drug delivery device may contain or be used with AMG 673 or another product containing a half-life extended (HLE) anti-CD33xanti-CD3 BiTE® (bispecific T cell engager) construct. In some embodiments, the drug delivery device may contain or be used with AMG 701 or another product containing a half-life extended (HLE) anti-B-cell maturation antigen (BCMA)xanti-CD3 BiTE® (bispecific T cell engager) construct. In some embodiments, the drug delivery device may contain or be used with AMG 757 or another product containing a half-life extended (HLE) anti-delta-like ligand 3 (DLL3)xanti-CD3 BiTE® (bispecific T cell engager) construct. In some embodiments, the drug delivery device may contain or be used with AMG 910 or another product containing a half-life extended (HLE) epithelial cell tight junction protein claudin 18.2xCD3 BiTE® (bispecific T cell engager) construct.

Although the drug delivery devices, assemblies, components, subsystems and methods have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

Those skilled in the art will recognize that a wide variety of modifications, alterations, and combinations can be made with respect to the above described embodiments without departing from the spirit and scope of the invention(s) disclosed herein, and that such modifications, alterations, and combinations are to be viewed as being within the ambit of the inventive concept(s).

1. A drug delivery device comprising:
a drug product container having at least one flexible wall and defining a cavity configured to contain a drug product;
a pressurized vessel containing a gas under pressure;
an urging member in working connection with the pressurized vessel such that, upon at least partial release of the gas under pressure, the urging member moves from a first portion of the drug product container to a second portion, thereby ejecting at least a portion of the drug product from the drug product container.

2. The drug delivery device in claim 1, wherein the urging member comprises at least one leaf spring configured to apply an urging force on a distal portion of the drug product container before applying the urging force on a proximal portion of the drug product container.

3. The drug delivery device in claim 1, wherein the urging member comprises at least one spring-loaded roller element configured to progressively apply mechanical pressure on the drug product container.

4. The drug delivery device in claim 3, further comprising guide slots for guiding the roller element.

5. The drug delivery device in claim 3, further comprising a torsion spring coupled with at last one of the roller element and the drug product container and configured to urge the drug product container with respect to the roller element such as to urge the drug product from the drug product container.

6. The drug delivery device in claim 1, wherein the urging member comprises a secondary inflatable bag is used to apply pressure progressively to the drug product container.

7. The drug delivery device in claim 6, wherein the secondary inflatable bag comprises a serpentine pattern used to apply pressure progressively to the drug product container.

8. A container for a drug product comprising:
a first wall and a second wall cooperating to define a cavity configured to contain a drug product; and
an outlet in fluid communication with the cavity to selectively permit the drug product to exit the cavity;
wherein at least a portion of at least the first wall or the second wall includes and/or is coupled with an anti-sealing component that resists sealing between the first wall and the second wall while the drug product exits the cavity.

9. The container as in claim 8, wherein the anti-sealing component includes a plurality of ridges or grooves.

10. The container as in claim 8, wherein only one of the first wall and the second wall includes the anti-sealing component.

11. The container as in claim 8, wherein the first wall and the second wall are a single, integrally formed component.

12. The container as in claim 8, wherein the first wall and the second wall are separate components that are coupled with each other.

13. The container as in claim 9, wherein the container is a sterile, flexible, non-pressurized IV bag.

14. A drug delivery system for delivering a drug product, comprising:
a drug product container containing a drug product;
a fluid path configured to receive the drug product from the drug product container; and
a drug delivery device positioned along and/or adjacent to the fluid path;
wherein the drug product container includes:
a first wall and a second wall cooperating to define a cavity configured to contain a drug product; and
an outlet in fluid communication with the cavity to selectively permit the drug product to exit the cavity;
wherein at least a portion of at least the first wall or the second wall includes an anti-sealing component that resists sealing between the first wall and the second wall while the drug product exits the cavity.

15. The drug delivery system as in claim 14, wherein the anti-sealing component includes a plurality of ridges or grooves.

16. The drug delivery system as in claim 14, wherein only one of the first wall and the second wall includes the anti-sealing component.

17. The drug delivery system as in claim 14, wherein the first wall and the second wall are a single, integrally formed component.

18. The drug delivery system as in claim 14, wherein the first wall and the second wall are separate components that are coupled with each other.

19. The drug delivery system as in claim 14, wherein the container is a sterile, flexible, non-pressurized IV bag.

20. The drug delivery system as in claim 14, wherein both first and second wall surfaces are treated with hydrophobic coatings or physical features to minimize any drug adhesion due to high surface tension of the drug.
